(19) United States
(12) Patent Application Publication
(10) Pub. No.: US 2012/0232335 A1
(43) Pub. Date: Sep. 13, 2012

(54) APPARATUS AND METHOD FOR MALE STIMULATION

(75) Inventors: Shane Eisenberg, Portland, OR (US); Colton Burgess, Portland, OR (US); Chad Gerson, Indianapolis, IN (US)

(73) Assignee: DIGITECH MEDIA LLC, Portland, OR (US)

(21) Appl. No.: 13/417,958

(22) Filed: Mar. 12, 2012

Related U.S. Application Data
(60) Provisional application No. 61/451,804, filed on Mar. 11, 2011.

(51) Int. Cl. A61F 5/00

(52) U.S. Cl. 600/38

(57) ABSTRACT

An apparatus and method for promoting ejaculation by a male human may include an input device and a video display apparatus in communication with, and receiving a control signal from, the input device. In operation, a control signal indicative of the motion of the input device may be transmitted to the video display apparatus. The motion data may then be used to control a frame rate for a video (or other content) associated with the video display.

### CROSS-REFERENCE TO RELATED APPLICATIONS

This application claims priority to U.S. Provisional Application No. 61/451,804, filed Mar. 11, 2011, the disclosure of which is incorporated herein in its entirety.

### FIELD OF THE INVENTION

An apparatus is disclosed for promoting ejaculation by a human male.

### BACKGROUND OF THE INVENTION

A high frequency of ejaculation (i.e., orgasm) activity by human males has been found to have health benefits for the individual. A 2004 study by Leitzmann M F, et. al., in the Journal of the American Medical Association indicated, for example, that men who ejaculated 21 or more times a month experienced a 33% lower risk of prostate cancer throughout their lifetimes compared with men who reported four to seven ejaculations a month. See Leitzmann M F, Platz E A, Stampfer M J, et al. Ejaculation Frequency and Subsequent Risk of Prostate Cancer. Journal of the American Medical Association 2004; 291:1578-86. PMID: 15069045. A 2003 study by Giles G G, et al, in BJU International indicated that men who averaged 4.6 to seven ejaculations a week were 36% less likely to be diagnosed with prostate cancer before the age of 70 than men who ejaculated less than 2.3 times a week on average. See Giles G G, Severi G, English E R, et al. Sexual Factors and Prostate Cancer. BJU International 2003; 92:211-16. PMID: 12887469. Data from still other studies indicated that positive health benefits related to the frequency of ejaculation included reduced stress and an improved immune system. Accordingly, the development and use of an apparatus and method that may promote male ejaculation would be advantageous to the health of the individual user.

### SUMMARY OF THE INVENTION

An apparatus and method for promoting ejaculation by a human male may include an input device, such as a human interface device (HID) or the like, and a video display apparatus in communication with, and receiving a control signal (e.g., packetized data signal) from, the input device.

An input device may include a device useful in the collection of sperm from a human male. The input device may be designed so that it may fit around a user's penis and also configured for reciprocal movement relative to the user. The input device may further include a motion sensor and a wireless transmitter. The motion sensor may include an accelerometer that collects motion information concerning the device. The wireless transmitter may include an RF microcontroller.

The video display apparatus may include a personal computer (including a CPU, a memory, a keyboard, a monitor, etc.) or similar device (e.g., ipad or like tablet device, iphone or like telecommunications device) that may operate to recognize a device connected to it physically via a USB port, such as a remote input device via a USB dangle, or a mouse or virtually via Bluetooth or like wireless connection. A video (or other content such as an animation, interactive game, virtual world, or camera system) may be stored in the video display apparatus and/or accessed by the user through that apparatus.

In operation, the velocity of the motion of an input device (e.g., the speed of movement of a device along a user's penis) may be measured by the accelerometer and transmitted by the RF microcontroller to a receiver (e.g., a USB dongle or Bluetooth equipped device) in communication with the video display apparatus. The velocity data may then be used to control a frame rate (in frames per second (fps)) for a video (or other content) associated with the video display. In this manner, an individual may use an input device to virtually interact with and control a video or like content on the video display apparatus. For example, where the video is a sexually explicit high-definition video featuring POV (point-of-view) scenes of a live actor, the input device may permit a user to virtually interact with the on-screen actor to facilitate ejaculation by the user.

### BRIEF DESCRIPTION OF THE DRAWINGS

Reference will be made herein to various figures, in which like reference numerals refer to like parts throughout, and in which:

FIG. 1 is an environmental perspective view of an embodiment of input device(s), including a wireless device, and video display apparatus constructed in accordance with the present invention;

FIG. 2 is an environmental view of an embodiment of input device(s), including wired devices, and video display apparatus constructed in accordance with the present invention;

FIG. 3 is a perspective view of an integrated circuit (IC) having a motion sensor and wireless transmitter positioned in a housing, the housing to be fitted to an input device,

FIG. 4 is a diagrammatic view of a scene selection and audio/video arrangement for an embodiment of a video presentation in accordance with the present invention;

FIG. 5 is a flowchart view showing an embodiment for a process of select and advancing an audio track(s) for a scene;

FIG. 6 is a flowchart view flowchart view showing an embodiment for advancing a video track for a scene;

FIG. 7 is side perspective view of one embodiment of a input device for use in connection with the present invention;

FIG. 8 is side perspective and partial cut-away view of the embodiment of the input device shown in FIG. 7;

FIG. 9 is a planar view of the embodiment of the input device shown in FIG. 7 taken along line 9-9 thereof;

FIG. 10 is a planar view of the embodiment of the input device shown in FIG. 7 taken along line 10-10 thereof; and

FIG. 11 is planar side view of another embodiment of a input device for use in connection with the present invention.

### DETAILED DESCRIPTION OF THE PREFERRED EMBODIMENTS

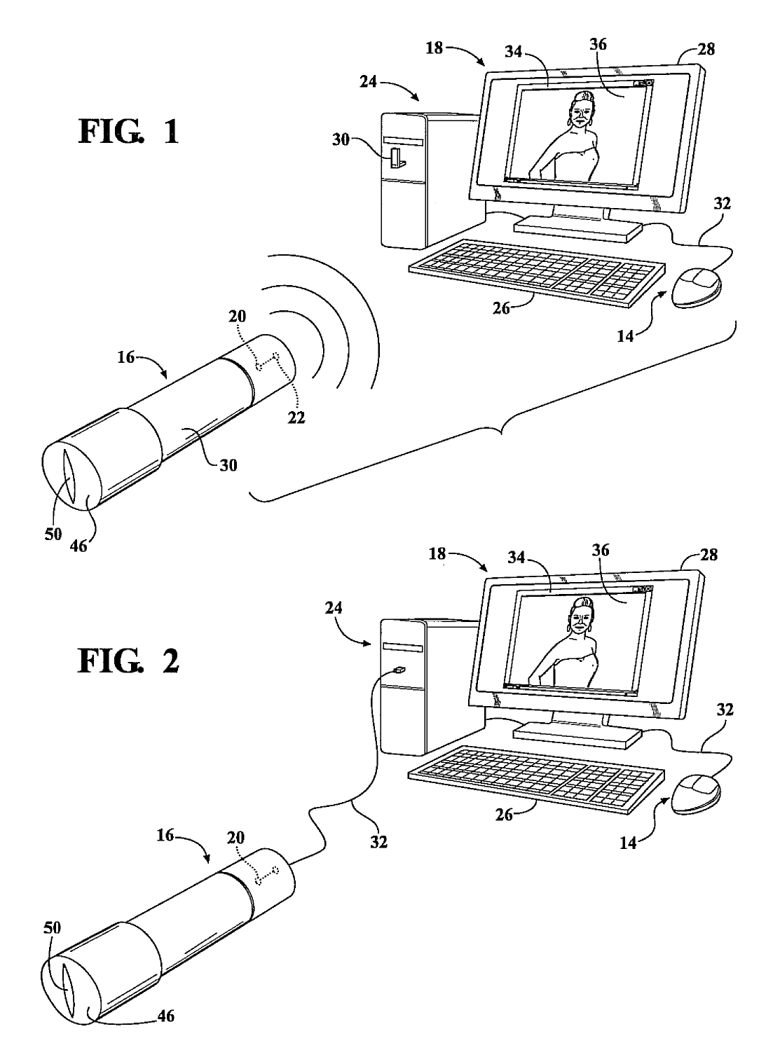

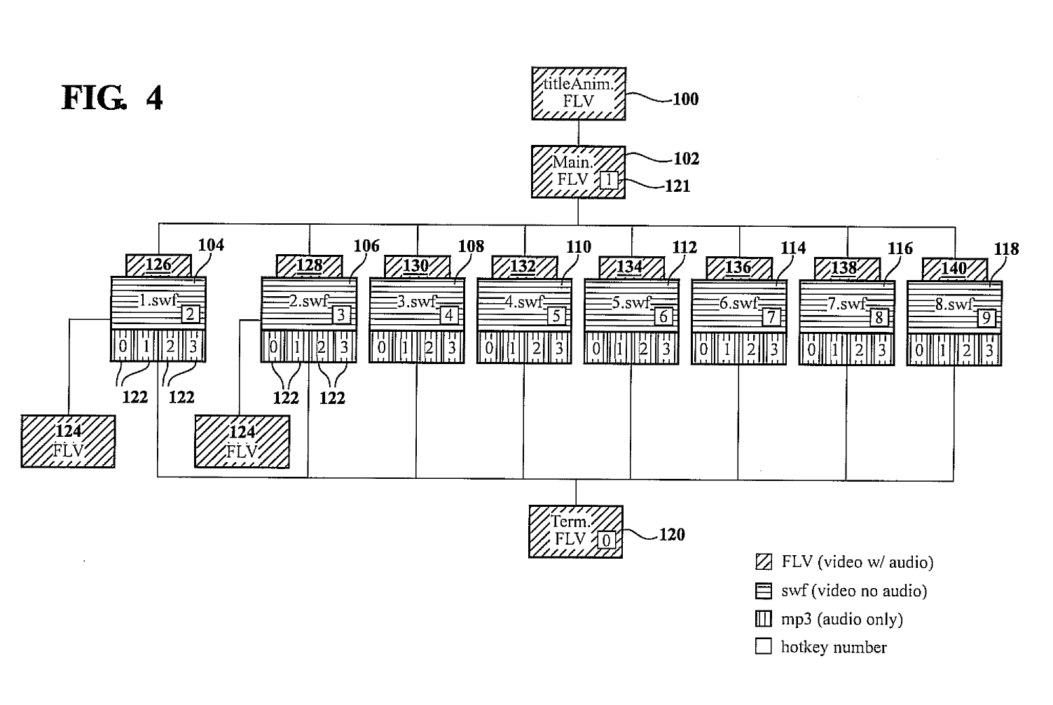

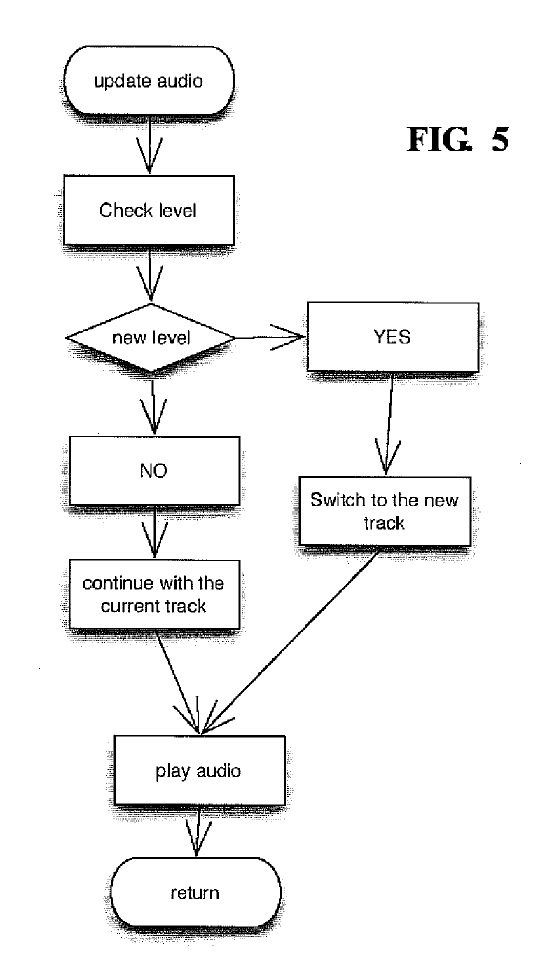

Referring now to FIGS. 1, 2, 4 and 5, an embodiment of apparatus 10 and method for promoting ejaculation by a human male may include an input device 12, such as a computer mouse 14 or a human interface device 16 (HID), and a video display apparatus 18 that may be in communication with, and receive a control signal (i.e., packetized data signal) from, the input device 12. A HID 16 may include a device to aid ejaculation by a human male and include a motion sensor 20 mounted to the device 16 and a transmitter 22 mounted to the HID 16 that is in communication with the motion sensor 20. The video display apparatus 18 may include a personal computer 20 (including a CPU, a memory, a keyboard 26, a monitor 28, etc.) or similar device (e.g., ipad or like tablet device, iphone or other electronic or telecommunication device) that may operate to recognize an input device 10 connected to it directly or remotely (e.g., via a USB port or Bluetooth etc.), such as a remote HID 16 via a receiver/USB dongle 30 or a mouse 14 or HID 16 via a cable 32. In operation, a control signal (e.g., packetized data signal) indicative of the position and/or velocity of the input device 10 (e.g., the movement of the mouse, or alternative a device 16 along a male user's penis) may be generated by the motion sensor 20 and transmitted by the transmitter 22 to a receiver 30 (e.g., a USB dangle) that is in communication with (or part of) the video display apparatus 18. The video display apparatus 18 (and/or a video player 34 thereon) may then function to control the playback speed (e.g., the frame rate) of a video presentation 36 (or other media content) associated with the video display apparatus 18 by speeding up or slowing down the video presentation 36 according to the position and/or velocity of motion of the input device 10. In short, the rapid movement of the input device 10 may result in faster playback (i.e., faster frame rate) and slower movement of the input device 10 may result in slower playback (i.e., slow frame rate). In addition, the video display apparatus 18 (and/or a video player 36 thereon) may function to select (see FIG. 5) one or more audio clips (see FIG. 4) associated with a video presentation 36 (or portion thereof) that corresponds to the speed/frame rate of playback.

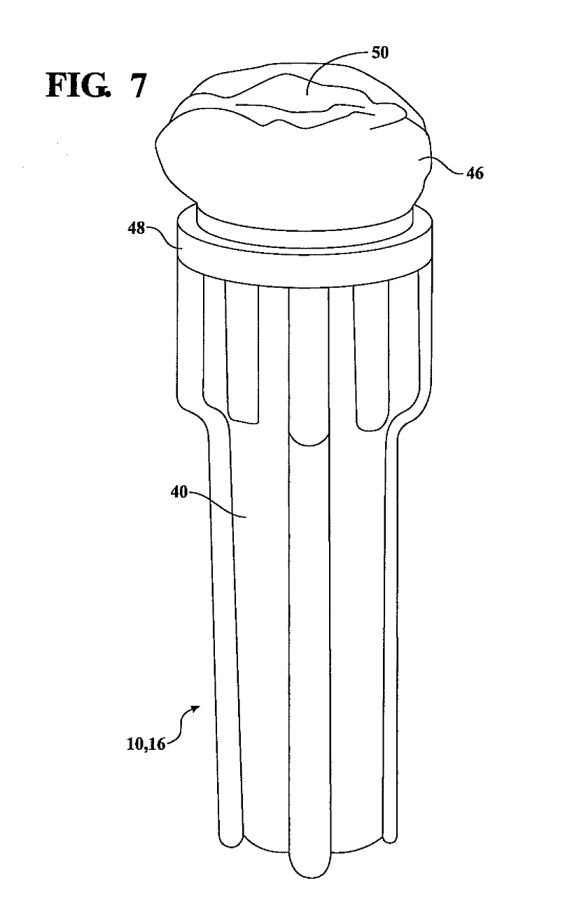

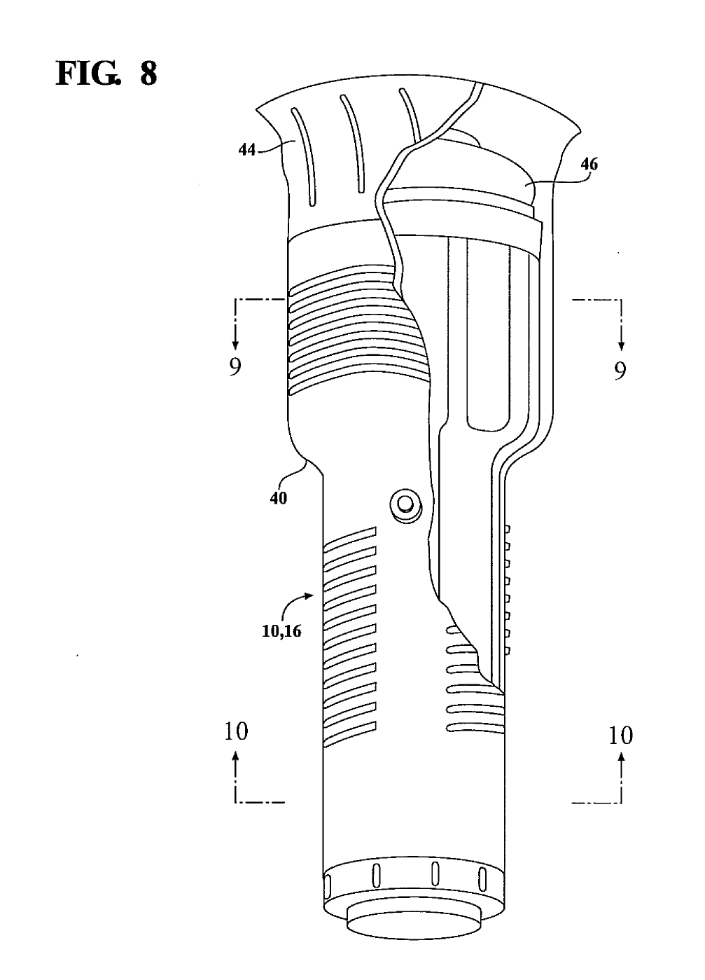

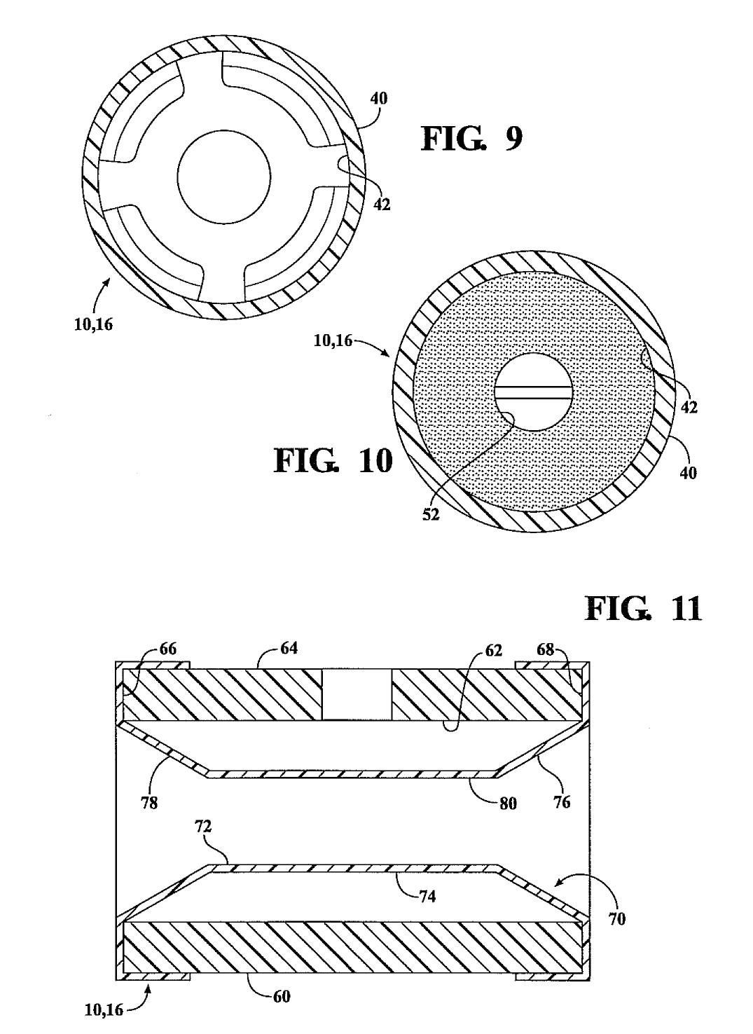

Referring now to FIGS. 1, 2 and 7-10, as mentioned supra, in one embodiment the input device 10 may be a human interface device 16 (HID), and specifically a device to aid ejaculation by a human male of the type disclosed (or similar thereto) in U.S. Pat. No. 5,782,818, the disclosure of which is incorporated herein by reference in its entirety. The device 10, 16 may, for example, include a shell 40 defining a generally tubular interior chamber 42 and having an exterior that may have an appearance similar to an ordinary device found in a public environment (e.g., a flashlight—see FIG. 8). The shell 40 may have a pair of ends, one open end and a closed (or closable end) and provide access to the chamber via at least one removable cap 44. An insert 46 may be positioned and/or mounted to one end of the shell 40. The insert 46 may be formed from an elastomeric gel and have the general tactile feel of human flesh. One end 48 of the insert 46 may have an opening 50 or aperture and it may be shaped to simulate a human body orifice (e.g. vagina, rectal orifice, mouth and/or lips). The insert 46 may also include a central passage 52 (see FIG. 10) sized to accommodate a human male sex organ (not shown) while providing at least a cushioning amount of the elastomeric gel. It will be appreciated that devices of the type just described are available under the name FLESHLIGHT®.

Referring now to FIGS. 1, 2 and 11, in another embodiment the input device 10 may be a human interface device 16 (HID), and specifically a device to aid ejaculation by a human male of the type disclosed (or similar thereto) in U.S. Pat. No. 6,149,580, the disclosure of which is also incorporated herein by reference in its entirety. In this embodiment, the device 10, 16 may include a core member 60 formed in a cylindrical configuration. The core member 60 may have an interior surface 62 and an exterior surface 64 with a first end 66 and an axially spaced second end 68. A surrounding member 70 may be formed in a cylindrical configuration and have interior 72 and exterior 74 surfaces with a first end 76 and an axially spaced second end 78. The surrounding member 70 may have a central section 80 positioned within the core member 60 and end regions stretched to be in contact with the exterior surface 64 of the core member 60 adjacent to the ends 66, 68 to thereby form a central cylindrical section and adjacent frustoconical sections.

In still further examples, the device 14 may be a product selected from the EGG, FLIP HOLE or CUP product lines produced by Tenga Co., Ltd. of Japan, or some other similar functioning devices known in the art.

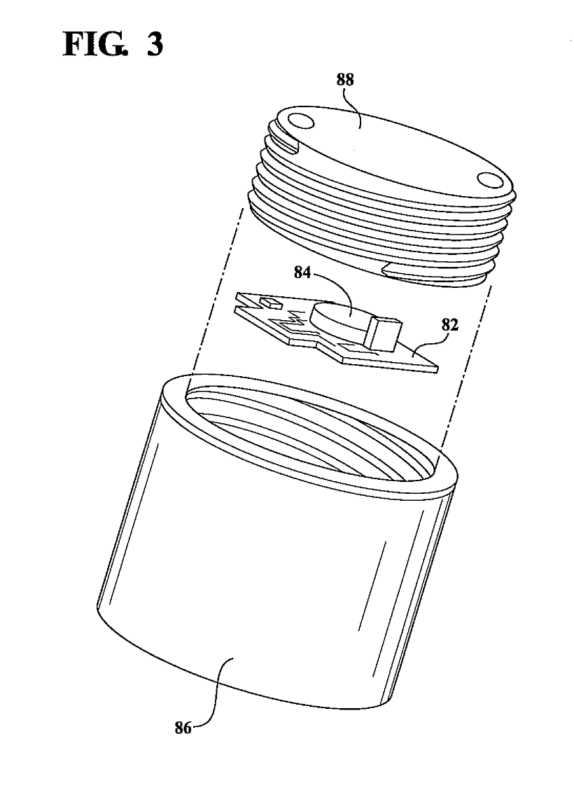

Referring now to FIGS. 1, 2 and 3, the motion sensor 20 may include a 3-axis digital accelerometer integrated circuit 82 powered by an on-board battery 84. The motion sensor 20, for example, may include a model ADXL345 accelerometer that is commercially available from Analog Devices. The ADXL345 is a small, thin, low power, 3-axis accelerometer with high resolution (13-bit) measurement at up to ±16 g. Digital output data is formatted as 16-bit twos complement and is accessible through either a SR (3- or 4-wire) or I²C digital interface. The ADXL345 has a high sensitivity of 3.9 mg/LSB enabling a measurement of inclination changes less than 1.0°. The ADXL345 may measure static acceleration of gravity for tilt-sensing applications, as well as dynamic acceleration resulting from motion or shock using microelectromechanical systems (MEMS) technology. MEMS include mechanical devices integrated into the device and interfaced electronically to other parts of the integrated circuit.

Referring now to FIGS. 1, 2 and 3, the transmitter 22 may include an RF microcontroller positioned on or integrated into the integrated circuit 82 that may be in communication with, and receive the control or data signal (e.g., packetized data signal) from, the motion sensor 20 (e.g., accelerometer) concerning the motion/speed/velocity of the input device 10, 16. The transmitter 22 may then function to transmit the signal to the video display apparatus 18.

Referring now to FIGS. 1, 2 and 3, the motion sensor 20 and transmitter 22 may be integrated into the input device 10, 16. Additional, or alternatively, the motion sensor 20 and transmitter 22 may be maintained in a separate housing 86 (closable by a threaded cap 88) that may be either fixed or removably mounted to the device 10, 16 (e.g., the end of the shell 40 opposite the insert 46). The use of a separate housing 86 permits the motion sensor 20 and transmitter 22 to be provided as an aftermarket unit for the FLESHLIGHT® (or similar devices) that may be mountable to the opposite end of that product. However, other methods of mounting or integrating the sensor 20 and transmitter 24 to an input device 10, 16 may also be used depending on the particular configuration of the input device 10, 16.

Referring now to FIGS. 1, 2 (and as mentioned supra) the video display apparatus 18 may include a personal computer 24 (including a CPU, a memory, a keyboard 26, a monitor 28, etc.) that may operate to recognize a signal transmitted from the input device 10, 14, 16 (that may be directly or remotely connected to the video display apparatus 18 via a USB port (such as a remote HID 16 via a receiver/USB dangle 30, or directly via cable 32 from a mouse 14 or HID 16)). A video presentation 364 (which may include a live action movie/video and/or other media content such as an animation, interactive game, virtual world, camera system or any combination thereof) may be stored in the video display apparatus 36 and/or remotely accessed and/or downloaded on or from a third party server by the user through that apparatus 18 as is known in the art. As mentioned supra, a receiver 30 for the video display apparatus 18 may also include an RF microcontroller or a USB dongle that is in communication with (e.g., plugged into) a USB port of the video display apparatus 18 (see FIG. 1). For example, where the receiver 30 is a USB dongle, the receiver 30 may receive the control signal (e.g., packetized data signal) from the transmitter 20 (e.g., RF microcontroller) associated with the motion of the device 10, 16 and pass that signal/data on to the video display apparatus 18 (e.g., PC 24).

Still referring now to FIGS. 1, 2, the video display apparatus 24 may also function to emulate a USB mouse and/or a device 14 described supra. However, it will be appreciated that once a USB device is connected to the video display apparatus 12 (e.g., PC) an enumeration process may occur as is known in the art. As part of the enumeration process the video display apparatus 18 (e.g., PC 24) may understand the capabilities of the input device 10, 16 and also the type of information to expect from the device 10, 16 during data transfer, and it may store a handle to this device in memory. The handle may be searched using the PID (product ID) and VID (vendor ID) of the USB device. The PID and VID of any commercial device embodying the apparatus described supra may already be stored. Then, as the connected device 10, 16 is identified, the handle to that device 10, 16 may be requested from the operating system (MS Windows®, Apple OS X, LINUX, or the like) for the video display apparatus 18 so that the video display apparatus 18 may communicate with the device 10, 18. As a result, device specific drivers may not be required. It will, however, be appreciated that a driver may be used depending on the particular needs of the user. Once a user connects a USB dongle 30 (see FIG. 1), or directly connects (see FIG. 2), to the video display apparatus 18 (e.g., PC), an operating system for the video display apparatus 18 may enumerate the associated device 10, 16 and continue about its work.

In operation, the transmitter 22 (e.g., an RF microcontroller) may receive a control or data signal generated by the motion sensor 20 (e.g., accelerometer) at a rate of about 200 hz. As a result, the bandwidth of the input device 10, 16 may be about 100 hz. It will also be appreciated that a Nyquist rate for sampling human motion is about 20 Hz and so a bandwidth of 100 hz will be sufficient to capture motion information from human activity. After receiving the control or data signal from the motion sensor 20, the transmitter 24 (e.g., an RF microcontroller) may then function to transmit that signal to the receiver 30 (e.g., a USB dongle) associated, and in communication with, the video display apparatus 18 at a baud rate of about 250 kbps over a 2.4 Ghz ISM free radio band. However, other wireless systems and networks such as Bluetooth, Zigbee and Wifi, may also be used to transmit the control or data signal to a receiver 30. Upon receipt of the control signal (e.g., packetized data signal) from the transmitter 22 (e.g., RF microcontroller), the receiver 30 may pass that signal/data on to the video display apparatus 18 (e.g., PC 24) at a rate of 10ms (i.e., 100 hz).

Referring now to FIGS. 1, 2 and 4, the video display apparatus 18 (e.g., PC) may also include a video player 34 or the like (not shown), such as a standalone desktop FLASH® or Abode AIR application, configured to be controlled by input device 10, 16 as described herein. A standalone desktop FLASH® application may function to load precompiled .swf movie scenes and play them at variable speed (i.e. variable playback frame rates). The SWFs of the interactive scenes may be compiled with AVM2 (ActionScript 3.0 setting in FLASH® CS). For example, where the movie/video is a sexually explicit high-definition video featuring POV (point-of-view) scenes of a live actor, the video player 34 may start with a .swf title animation and proceed to display a movie clip.

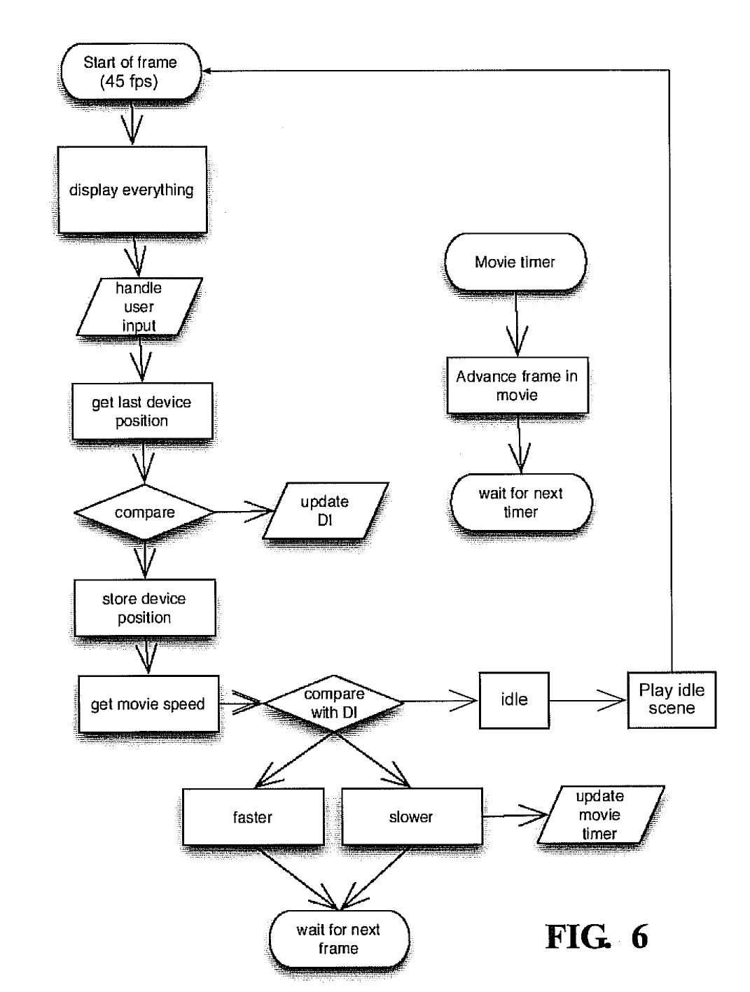

Referring now to FIGS. 1, 2 and 6, a velocity of the input device 10, 16 may be measured using a timer and comparing a current position of an input device 10, 16 to a previous position. This velocity may be regarded as device intensity DI. Relative to DI and the current speed of the video presentation 36, the video presentation 36 may be either sped up or slowed down. There may also be a separate delay timer that advances the movie. Timer delay may vary from 1000/0 (infinity or stop) to 1000/60 (60 hertz or full speed) depending on the DI. It will also be appreciated that the video display apparatus 18 may be configured to playback a video presentation 36 to play at “normal” speed when the frame rate is about 45 frames per second (fps) since a presentation 36 otherwise indexed for 60 fps movie would be played at a slower speed—and result in the video presentation 36 having a “slow motion” appearance. Additionally, or alternatively, where operation of the input device 10, 16 would result in a fast rate of playback (e.g., greater than 45 fps) the video display apparatus 18 (or player 34 thereon) may be configured to “skip” one or more frames to better simulate the faster motion of the input device. More specifically, the video display apparatus 18 may monitor the maximum frame rate being achieved and then automatically skip frames to achieve the appropriate fastest speed in fps. This “skip” technique may be used, for example, where the video display apparatus is of a type that cannot achieve 60 fps playback.

Referring now to FIGS. 1, 2 and 4-6, in one embodiment the video presentation 36 may include a plurality of separate video clips (e.g., a title clip 100, a main clip 102, eight interactive scenes, non-interactive introductions to each interactive scene 126-140, non-interactive “idle scenes” corresponding to each interactive scene 124, and a termination clip 120. The title animation 100, main clip 102, the non-interactive introductions 126-140 to each interactive scene 104-118, the idle scenes 124 corresponding to each interactive scene 104-118, and the termination 120 clip may be FLV (Flash video) clips. The interactive scenes 104-118 may be formatted in .swf compression and featuring a performer in different acts and/or positions. The playback speed of these interactive clips may be controlled by the speed at which the input device 10, 16 and may vary from 0-60 frames per second (fps). As mentioned supra, the frame rate changes according to a user's speed of motion of the input device 10, 14. Thus, as a user speeds up, so does the frame rate (speed of on-screen activity), when a user slows down, the frame rate decreases, and when a user stops, so does the video. As a result, the user has the impression that his or her own actions (i.e., speed) are being displayed on the video display apparatus 18, 34. In operation, the various scenes may be accessed in chronological order (i.e., title 100, main 120, interactive 1-8 (104-118) and termination 120). Additionally, or alternatively, each scene may be directly accessed using “hotkeys” 121. In one embodiment, when the user selects an interactive scene 104-118, the presentation may first play a non-interactive introductory scene 126-140 corresponding to the chosen interactive scene 104-118 showing, for example, the performer preparing for the action of the associated interactive scene (e.g., 104-118)(i.e., assuming a particular position and/or speaking provocatively). When the non-interactive introductory scene 126-140 completes, the chosen interactive scene 104-118 may then automatically begin to play, or if the user so desires, he may skip this non-interactive introductory scene 126-140 at any time while it is playing by pressing the space bar (or like entry) on his computer's keyboard 26.

Still referring now to FIGS. 1, 2, 4-6, each interactive scene (e.g., 104-118) may also include one or more independently controllable audio tracks 122 that are selectively played according to the measured speed/velocity of the input device 10, 16. As shown, each scene may include four separate audio tracks 122. However, it will be appreciated that each scene (e.g., 104-118) may include 2, 3, 4, 5 or 6 tracks 122 or any number of tracks in a range between 1-50. The “1” track 122 may be played at slow speed, “2” at medium speed, and “3” at fast speed. Each track 122 may also represent a different level of intensity in terms of the on-screen performer's response. Accordingly, track 1 may be the performer breathing, track 2: moaning, and track 3: screaming. An optional fourth track, a “0” track, may be played automatically if the input device 10, 14, 16 is idle for a predetermined period of time (e.g., 5 seconds). The “0” track 122 may, for example, be an audio clip of the performer encouraging the user to continue. In an alternative embodiment, if the input device 10, 14, 16 is idle for a predetermined period of time (e.g., 1-5 seconds) the interactive scene 104-118 may change to one of a plurality of additional, but non-interactive “idle scenes” 124 (in e.g., FLV format), in which the performer or animation, etc. coaxes a user to continue or delivering some other message. In addition, while only two non-interactive “idle scene” 124 are shown (see FIG. 4) it will be appreciated that each interactive scene (e.g., 104-118) may have one or more of its own dedicated associated non-interactive “idle scenes” 124 to be used for this purpose, that corresponds to the position or activity of the performer/animation in the related interactive scene (e.g., 104-118). When that non-interactive scene 124 is finished the related interactive scene (e.g., 104-118) may resume immediately or the user can resume moving the input device 10, 14 while the non-interactive “idle scene” 124 is playing to skip immediately back to the related interactive scene (e.g., 104-118).

Having described an embodiment of the invention, various other embodiments will become apparent to those of skill in the art that do not depart from the scope of the claims.

1. An apparatus comprising:
a human interface device and a video display apparatus, the human interface device including a generally cylindrical shell, a motion sensor, a wireless transmitter, and an insert, the shell defining an interior chamber and having at least one open end, the insert being removably mounted to the open end and having a face defining an aperture, the motion sensor operating to generate a control signal indicative of the movement of the shell, and the wireless transmitter being in communication with the motion sensor and transmitting the control signal to the video display apparatus, and the video display apparatus including a receiver and a video presentation, the receiver receiving the control signal from the wireless transmitter and advancing the video presentation in response to the control signal.

2. The apparatus of claim 1, wherein the insert comprises an elastomeric insert.

3. The apparatus of claim 2, wherein the elastomeric insert comprises an elastomeric gel.

4. The apparatus of claim 3, wherein the face of the insert is configured to simulate the shape of a human body orifice.

5. The apparatus of claim 4, wherein the insert comprises a central passage, the aperture in the face of the insert defining an opening to the central passage.

6. The apparatus of claim 1, wherein the motion sensor comprises an accelerometer.

7. The apparatus of claim 6, wherein the accelerometer comprises a 3-axis digital accelerometer.

8. The apparatus of claim 1, wherein the wireless transmitter comprises a radio frequency microcontroller.

9. The apparatus of claim 1, wherein the video display apparatus comprises a telecommunications device.

10. An apparatus comprising a shell, a motion sensor, a wireless transmitter, and an insert positioned on the shell, the shell defining an interior space and having at least one open end, the insert being an elastomeric insert positioned on the open end, the motion sensor including an accelerometer and operating to generate a control signal indicative of the movement of the shell, and the wireless transmitter being in communication with the motion sensor and operating to transmit the control signal.

11. The apparatus of claim 10, wherein the accelerometer comprises a 3-axis digital accelerometer.

12. The apparatus of claim 10, wherein the wireless transmitter comprises a radio frequency microcontroller.

13. The apparatus of claim 10, wherein the insert is configured to simulate the shape of a human body orifice.

14. A method comprising:
providing an input device having a motion sensor, the motion sensor operating to measure a velocity of the input device and transmit a control signal indicative of the velocity of the input device;
providing a video display apparatus in communication with the input device and operable to receive the control signal, the video display apparatus including a video presentation, the video presentation including at least one video track and at least one audio track;
receiving by the video display apparatus the control signal from the input device; and
the video display apparatus advancing the video presentation at a frame rate proportional to the velocity of the input device.

15. The method of claim 14, wherein the video presentation includes an interactive video scene and a plurality of audio tracks associated with the interactive video scene and the video display apparatus playing a predetermined one of the plurality of audio tracks corresponding to a predetermined frame rate.

16. The method of claims 14, wherein the input device is a device to aid ejaculation by a male human.

17. The method of claim 15, where the input device is a human interface device.
